(19) United States
(12) Patent Application Publication
(10) Pub. No.: US 2025/0017657 A1
(43) Pub. Date: Jan. 16, 2025

(54) SYSTEM AND METHOD FOR DETERMINING OPTIMAL PLACEMENT OF HIP IMPLANTS

(71) Applicants: Smith & Nephew, Inc., Memphis, TN (US); Smith & Nephew Orthopaedics AG, Zug (CH); Smith & Nephew Asia Pacific PTE. Limited, Singapore (SG)

(72) Inventors: Alessandro Navacchia, San Clemente, CA (US); Elizabeth A. Duxbury, San Clemente, CA (US); David W. Rister, Nesbit, MS (US)

(73) Assignees: Smith & Nephew, Inc., Memphis, TN (US); Smith & Nephew Orthopaedics AG, Zug (CH); Smith & Nephew Asia Pacific PTE. Limited, Singapore (SG)

(21) Appl. No.: 18/711,352

(22) PCT Filed: Nov. 3, 2022
(86) PCT No.: PCT/US2022/048821

§ 371 Date: May 17, 2024

Related U.S. Application Data
(60) Provisional application No. 63/291,783, filed on Dec. 20, 2021.

(51) Int. Cl. A61B 34/10; A61B 90/00; A61F 2/32; A61F 2/46

(52) U.S. Cl. CPC A61B 34/10; A61F 2/32; A61B 2034/104; A61B 2090/364; A61F 2002/4633

(57) ABSTRACT

Disclosed herein is a system and method providing a tool for use by a surgeon, either preoperatively or intra-operatively, to visualize the effect of and select optimal design and placement of an implant used in a total hip replacement surgical procedure on both the range-of-motion of the patient's leg and the jump distance of the implant. This will assist the surgeon in determining the optimal selection and placement of the implant to provide the best outcome for the patient.

### RELATED APPLICATIONS

This application claims the benefit of U.S. Provisional Patent Application No. 63/291,783, filed Dec. 20, 2021, the contents of which are incorporated herein in their entirety.

### FIELD OF THE DISCLOSURE

The present disclosure relates generally to orthopedic devices and procedures and, more particularly, to a total hip replacement procedure which includes systems and methods for assisting the surgeon to visualize and select the best position of the implant to optimize the tradeoff between range-of-motion and stability and for visualizing the effects of and selecting the optimal placement of the lipped portion of the cup liner of the implant.

### BACKGROUND

Orthopedic implants are well known and commonplace in today's society. Orthopedic implants may be used, for example, to stabilize an injury, to support a bone fracture, to fuse a joint, and/or to correct a deformity. Orthopedic implants may be attached permanently or temporarily and may be attached to the bone at various locations, including being implanted within a canal or other cavity of the bone, implanted beneath soft tissue and attached to an exterior surface of the bone, or disposed externally and attached by fasteners such as screws, pins, and/or wires. Some orthopedic implants allow the position and/or orientation of two or more bone pieces, or two or more bones, to be adjusted relative to one another.

One such orthopedic implant is used during a total hip replacement procedure to replace a hip joint. The hip joint is a frequent place for joint damage and/or injury. Implants used in total hip replacement surgeries may include an elongated insertion portion, referred to herein as the “stem portion”, which can be at least partially inserted into the intramedullary canal of the patient's proximal femur, and a “cup” portion, which is embedded into the pelvis of the patient. The cup portion of the implant accepts the head of the stem portion to allow rotation of the stem portion with respect to the hip bone.

In some instances, excess rotation of the stem portion of the implant with respect to the hip bone and the cup portion of the implant can cause a dislocation. This often occurs, for example, when the stem portion contacts the lip of the cup portion, causing a lateral translation of the center of the femoral head. The lateral translation of the femoral head in excess of a particular distance, based on the geometry of the implant, may result in a dislocation. This distance is known as the “jump distance” and is defined as the distance the femoral head needs to travel to dislocate. The jump distance is illustrated in FIG. 1, showing the lateral translation of the center of the femoral head causing a dislocation. The length of translation is the jump distance.

During the total hip replacement procedure, the surgeon must cut various muscles and ligaments of the hip capsule, which tends to weaken those muscles and ligaments, limiting their ability to contain the hip joint in the hip capsule post-surgery. The weaker muscles and ligaments in the hip capsule often allow for an easier dislocation. To address this, the cup portion of the implant may be provided with a liner which may have a lipped portion to enhance stability of the joint and to reduce the probability of a dislocation.

When the cup is implanted into the hip bone, the lipped portion of the implant is oriented toward the direction of potential dislocation. For example, the lipped portion may be placed posteriorly for a total hip replacement implanted using a posterior surgical approach to reduce posterior dislocation risk, or anteriorly for anterior or lateral surgical approaches, to reduce anterior dislocation risk. Cups having lipped liners increase the jump distance, and therefore the stability of the implant, in the direction of the orientation of the lipped portion of the liner. However, the lipped liners also decrease the range-of-motion of the implant, because the stem may contact the lipped portion of the liner earlier in the rotation than with a non-lipped liner.

One goal of the surgeon during the total hip replacement surgery is to maximize both the range-of-motion and the jump distance. As such, there is a trade-off between range-of-motion and the probability of a dislocation, as dictated by the jump distance.

In certain surgeries, the hip implant may be provided with a cup liner having a lipped portion. One variable having an effect on the range-of-motion and the jump distance is the positioning of the lipped portion of the liner. For example, as shown in FIG. 2, during a posterior surgical approach, the lipped portion of the liner may be placed in a posterior-inferior position (8 o'clock), in a posterior position (9 o'clock) or in a posterior-superior position (10 o'clock), or, with some implants, at any position in between these discrete positions. The positionings of the lipped portion of the cup shown in FIG. 2 are exemplary and specific to the posterior surgical approach. Other surgical procedures using either an anterior or lateral surgical approach would position the lipped portion of the cup at different orientations with respect to the clock face shown in FIG. 2.

Currently, pre-operative planning applications and tools do not provide a way for the surgeon to visualize the effect of the positioning of the implant on the range of motion and the jump distance. Further, there is currently no application or tool to visualize the effect of the positioning of the lipped portion of the cup on either the range-of-motion or the jump distance. As such, there is no way for the surgeon, other than through an intuitive positioning of the implant and the lipped portion of the cup, to be able to optimize the tradeoff between the range-of-motion and the jump distance. Therefore, it would be desirable to provide to the surgeon a means of visualizing the effect of the positioning of the implant and the lipped portion of the cup liner on both the range-of-motion and the jump distance, and, in some instances, to provide recommendations as to the optimal placement.

### SUMMARY OF THE DISCLOSURE

Disclosed herein is a system and method providing a tool for use by a surgeon, either pre-operatively or intra-operatively, to visualize the effect of the orientation of the lipped portion of the cup liner on both the range-of-motion and the jump distance and identify the lip orientation that provides optimal tradeoff between jump distance and range-of-motion when other modifiable or non-modifiable parameters of the entire episode of care (e.g., patient's spinopelvic mobility, pre-selected cup placement, native femoral version) are taken into consideration. This will assist the surgeon in determining the optimal placement of the lipped portion of the cup liner to provide the best outcome for the patient.

In a first example, range-of-motion profiles and jump distance profiles for various orientations of the implant are visualized using a 2D or 3D visual representation of profiles of the implant's range-of-motion and jump distance for possible directions of impingement (of the stem portion of the implant on the edge of the cup portion of the implant). For example, in some examples, the range-of-motion can be visualized on diagrams including a coordinate system showing rotation of the leg of the patient around the X-axis, which approximates flexion/extension of the leg, and the Y-axis, which approximates abduction/adduction of the leg, as shown in FIG. 3, wherein the horizontal axis represents motion of the patient's leg about the Y-axis and the vertical axis represents motion of the patient's leg about the X-axis. One such exemplary coordinate system is shown in FIG. 4. To assess the best choice of placement of the implant with respect to range-of-motion, in this exemplary example, a 2D or 3D visualization of the kinematics of a hip indicating the desired approximate range-of-motion of the patient's leg is overlayed on the range-of-motion profiles. An assessment can be made with respect to each possible direction of impingement on the coordinate system to determine the best orientation of the implant such that the desired motion of the leg does not fall outside on the range-of-motion profiles, which implies impingement occurrence. The jump distance can be assessed in the same manner for each possible direction of impingement to determine the best orientation of the implant, based on which orientation provides the largest jump distance for relevant possible direction of impingement. Once both the range-of-motion and the jump distance profiles are visualized, it can then be determined from the visualization which orientation of the implant optimized the tradeoff between the range-of-motion and jump distance.

Alternatively, an optimization algorithm can be utilized to minimize a cost function and determine optimal lip orientation, given non-modifiable pre-operative and intra-operative parameters. For example, the cost function might penalize lip orientations that prevent selected hip range-of-motion rotations by more than 10 degrees, and/or maximize jump distance for a given rotation deemed most dangerous for the patient (e.g., combined flexion and internal rotation of the femur for patients operated with a posterior approach).

In a first example, a method includes visualizing a range-of-motion for a plurality of orientations of a hip implant based on various combinations of placement options for a cup portion and a stem portion of the implant and visualizing a jump distance for a plurality of orientations of the implant. (In this example, placement of the cup portion and stem portion are also modifiable factors together with lip orientation and an optimization algorithm can be utilized to find optimal position of the three components (i.e., cup, stem, and liner lip) that maximizes a pre-selected relationship between range-of-motion and jump distance (i.e., cost function)).

In any preceding or subsequent example, the method further includes providing 2D or 3D visualizations of the implant's range-of-motion and jump distance profiles for various possible directions of impingement.

In any preceding or subsequent example, the method further includes providing a visualization of a desired range-of motion of the patient's leg as an overlay on the range-of-motion.

In any preceding or subsequent example, the method further includes visualizing the range-of-motion by plotting one or more profiles representing one or more ranges-of-motion on a coordinate system having a vertical axis defining rotation of the patient's leg around an X-axis and a horizontal axis defining rotation of the patient's leg around a Y-axis.

In any preceding or subsequent example, the method further includes wherein the ranges-of-motion of a patient's leg are visualized as a polygon overlaid on the coordinate system showing the kinematics of a hip based on a positioning of the implant, wherein the plurality of profiles represent different orientations of the implant.

In any preceding or subsequent example, the method further includes determining acceptable orientations of the implant by determining whether the visualization of the ranges-of-motion of the patient's leg representing the kinematics of the hip extends outside of one or more of the profiles representing the range-of-motion.

In any preceding or subsequent example, the method further includes determining the positioning of the cup portion and stem portion of the implant for various cup inclinations, cup anteversions and stem anteversions.

In any preceding or subsequent example, the method further includes plotting a plurality of profiles representing the jump distance on a coordinate system having a vertical axis corresponding to rotations of the patient's leg about an anterior-posterior axis of a cup portion of the implant and a horizontal axis corresponding to rotations of the patient's leg about a medial-lateral axis of the cup axis of the cup portion of the implant.

In any preceding or subsequent example, the method further includes selecting, for each quadrant of the coordinate system, a preferred range-of-motion and jump distance profile such that the range-of-motion and jump distance are maximized for possible directions of impingement.

In any preceding or subsequent example, the method further includes receiving an input of a positioning of the implant.

In any preceding or subsequent example, the method further includes receiving an indication of a cup anteversion, a stem anteversion and a cup inclination and providing a visualization of effect of the placement of a lipped portion of the cup liner on the range-of-motion and jump distance based on the received indications.

In a second example, a system includes a processor and software that, when execute by the processor, causes the system to implement any preceding or subsequent example.

In a third example, the method of any preceding or subsequent example further includes providing a plurality of surgical recommendations based on various possible positionings of the implant.

In any preceding or subsequent example, the method further includes providing a recommendation, based on the received input, for orientation of the lipped portion of the cup liner.

In any preceding or subsequent example, the method further includes analyzing the indicated implant positioning and providing a recommendation of the orientation of the lipped portion of the cup liner via a mathematical algorithm or a machine learning model.

In a fourth example, a system includes a processor and software that, when execute by the processor, causes the system to implement any preceding or subsequent example.

Examples of the present disclosure provide numerous advantages. For example, the described system and method provides a surgeon performing total hip replacement procedures with a tool for visualizing the orientation of the implant and the effect of various orientations on the patient's range-of-motion. Additionally, the system and method provide the further advantage of showing the effect of the orientation of the lipped portion of the cup liner on the jump distance of the implant and its effect on range-of-motion. A further advantage is provided by a pre-operative portion of the system and method which shows the surgeon different options for orientation of the cup and stem components of the implant and the resulting impact on jump distance and the patient's range-of-motion. In a second example, the system and method provide the additional advantage of providing recommendations as to the orientation of the implant based given desired range-of-motion.

Further features and advantages of at least some of the examples of the present disclosure, as well as the structure and operation of various examples of the present disclosure, are described in detail below with reference to the accompanying drawings.

### DEFINITIONS

As used herein, the term “visualize” or “visualization” shall be construed to include any means of conveying the required information on a display device, including, for example, a text description, a chart, a graph (for example, on a coordinate system), an illustration, a figure, a drawing, a model, an image, an animation, a video, or any combination thereof.

As used herein, the term “optimize” or “optimization” in the context of providing a recommendation, means selection of parameters that are optimal based on certain specified criteria. In an extreme case, optimization can refer to selecting optimal parameter(s) based on data from the entire episode of care, including any pre-operative data, the state of CASS data at a given point in time, and post-operative goals. Optimization may be performed using historical data, such as data generated during past surgeries involving, for example, the same surgeon, past patients with physical characteristics similar to the current patient, or the like.

### BRIEF DESCRIPTION OF THE DRAWINGS

By way of example, specific exemplary examples of the disclosed system and method will now be described, with reference to the accompanying drawings, in which:

FIG. 1 is a schematic diagram of an implant used during a total hip replacement procedure illustrating the jump distance.

FIG. 2 is an illustration of a hip bone showing possible orientations of the lipped portion of the cup liner of the implant during a surgery using a posterior surgical approach.

FIG. 3 is an illustration of the X-and Y-axes used for profiling the range of motion and the jump distance.

FIG. 4 is a diagram illustrating the ranges of motion for orientations of the lipped portion of the cup liner, as well as a polygon showing selected kinematics of the hip joint.

FIG. 5 shows a plurality of plots of the diagram of FIG. 4 for various positionings of the cup portion and the stem portion of the implant, the plots changing for increasing cup inclinations and increasing cup anteversions for a constant stem anteversion.

FIGS. 6(A-D) shows a series of diagrams visualizing, for various stem anteversions, orientations providing the best range-of-motion for various combinations of the cup inclination and cup anteversion.

FIG. 7 is a visualization showing the lip orientation providing the largest jump distance for different sets of impingement directions.

FIG. 8 is a visualization showing the orientation providing the largest range-of-motion for different sets of impingement directions.

FIG. 9 is a series of diagrams showing range-of-motion plots on the left and jump distance plots on the right, with a dotted arrow indicating a hypothetical direction of impingement.

FIGS. 10A-12 show exemplary alternate visualizations for the range-of-motion and the jump distance.

FIG. 13 is a flow diagram showing a pre-operative workflow in accordance with the described examples.

FIG. 14 is a flow diagram showing an intra-operative workflow in accordance with the described examples.

FIG. 15 depicts an operating theatre including an illustrative computer-assisted surgical system with which various examples of the present disclosure could be used.

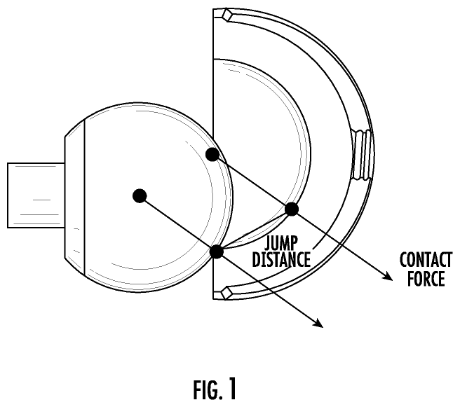

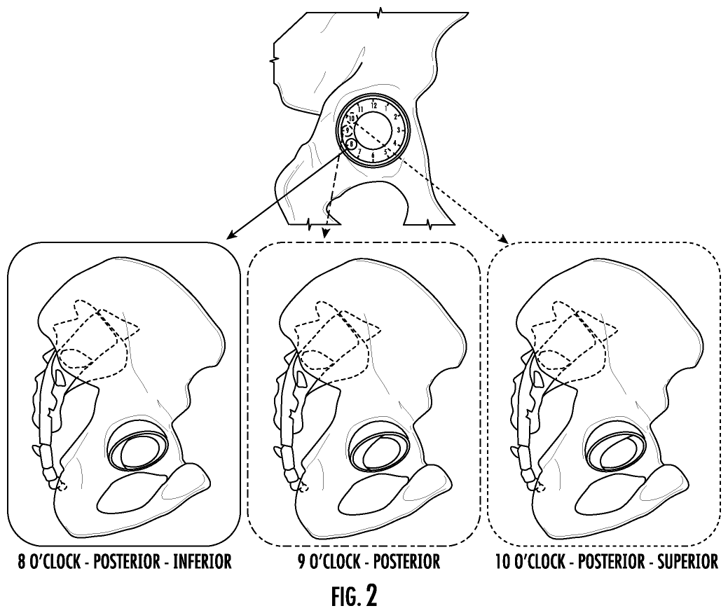

### DETAILED DESCRIPTION

The system and method disclosed herein provides information to a surgeon during the pre-operative and intra-operative phases of a joint replacement surgery which will assist the surgeon in determining the optimal positioning of the implant. Note that, although the disclosure is explained in terms of a total hip replacement procedure, all or portions of the present disclosure may be applicable to any joint replacement procedure using a ball and socket type of implant.

The system and method visualizes the effects of the placement of the implant on the range-of-motion and jump distance. The system may be used either pre-operatively, to advise the surgeon of the optimal placement given various combinations of cup inclination, cup anteversion and stem anteversion, or intra-operatively after the surgeon has determined an acceptable positioning of the implant based on the patient's anatomy.

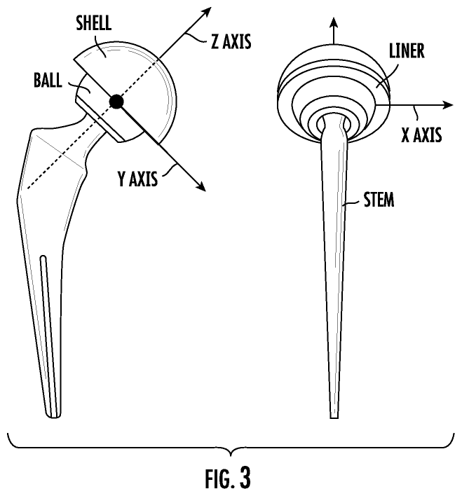

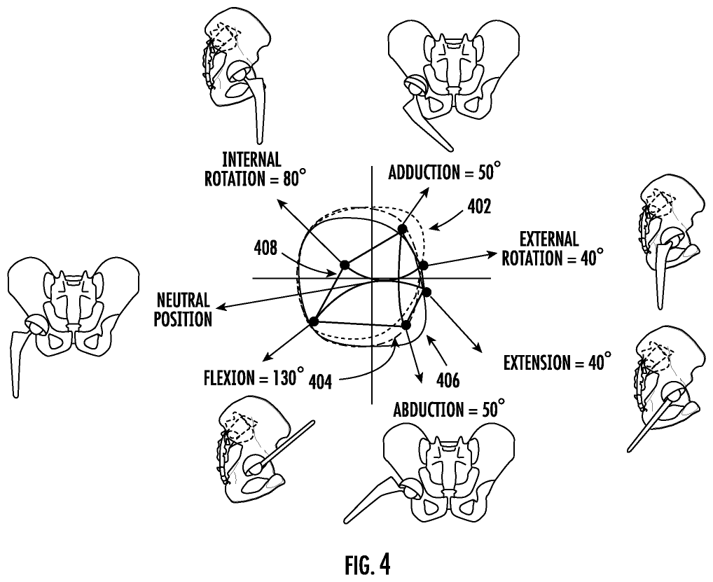

FIG. 4 shows a diagram which is the basis for determining which orientation of the implant provides the best range-of-motion. The diagram includes a coordinate system wherein the vertical axis represents rotation of the patient's leg about the X-axis shown in FIG. 3 (flexion/extension) and wherein the horizontal axis represents the rotation of the patient's leg about the Y-axis shown in FIG. 3 (abduction/adduction). For a posterior surgical approach, three profiles are provided which show the range-of-motion in both about both the X-and Y-axes. Profile 402 shows the range-of-motion when the lipped portion of the cup liner is placed in the posterior-superior (10 o'clock) position; profile 404 shows the range-of-motion when the lip of the cup liner is placed in the posterior (9 o'clock) position; profile 406 shows the range-of-motion when the lip of the cup liner is placed in the posterior-inferior (8 o'clock) position. The larger the area within the profile, the larger the range-of-motion.

Polygon 408 represents the kinematics of a hip showing the desired range-of-motion. The positioning of polygon 308 will vary based on the specific position in which the implant is placed (i.e., cup inclination, cup anteversion and stem anteversion). When polygon 408 is completely within one of profiles 402, 404, 406, there will be no impingement of the stem on the lip of the cup. However, if the polygon 408 touches a profile or extends outside of the profile, impingement will occur, thus limiting the range-of-motion.

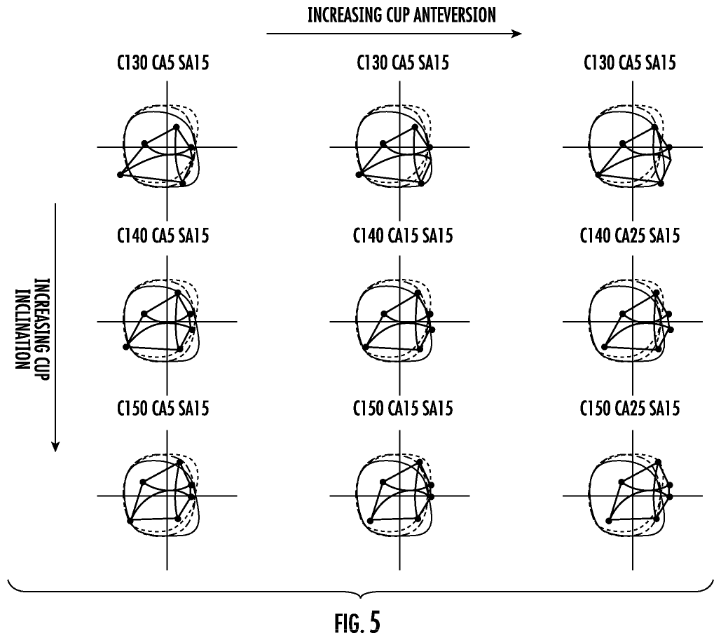

FIG. 5 shows how this diagram is used. The 9 plots shown in FIG. 5 show different positions of polygon 508 depending upon the placement of the lipped portion of the cup liner, with the angle of the cup anteversion increasing from left to right (i.e., 5°, 15° 25°) and the angle of the cup inclination increasing from top to bottom (i.e., 30°, 40°, 50°). This exemplary set of plots is for an implant placement having a stem anteversion of 15°. A separate set of plots would be presented for other positionings of the stem having differing stem anteversions. The exemplary set of plots shown in FIG. 5, and other sets of plots for other stem anteversion placements would be most useful for the surgeon pre-operatively. If the system is used intra-operatively, the surgeon may specify one or more of the cup inclination, the cup anteversion or the stem anteversion and be presented with plots showing the optimal placement of the lip portion of the cup when the indicated placements are used.

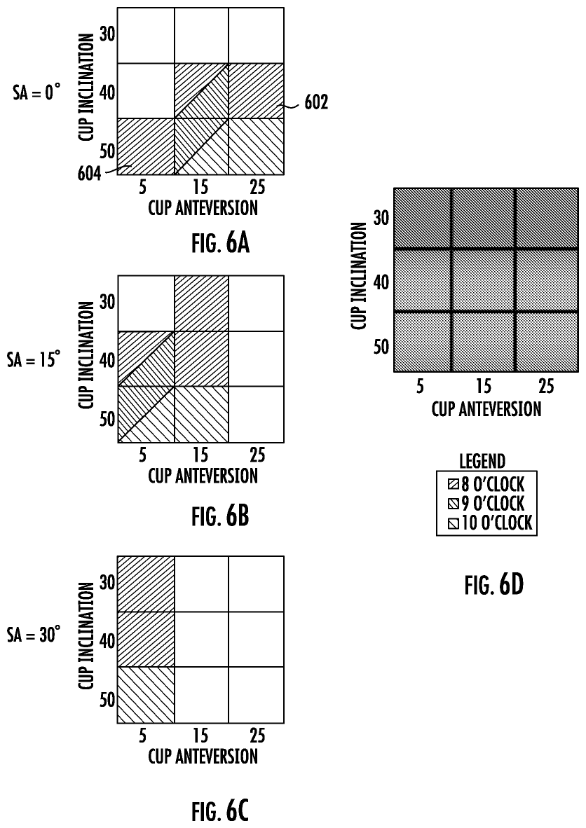

FIGS. 6(A-D) shows the cup liner orientation that provides a better range-of-motion, given cup and stem placement. For example, for stem anteversion=0° (FIG. 6A) the posterior-inferior squares 602, 604) orientation of the liner has better range-of-motion with cup inclination ˜40° and cup anteversion ˜25° (square 602), and with cup inclination ˜50° and cup anteversion ˜5° (square 604). White squares indicate that there is no liner orientation (among the three orientations taken into account here) that provides acceptable range-of-motion with those cup and stem angles. When a square has two colors, it means that both liner orientations are equivalent. FIGS. 6B and 6C show the results for stem anteversions of 15° and 30°, respectively. FIG. 6D indicates that, overall, the orientation shown by the darkest areas is better with low cup inclinations, (˜30°-40°), the liner orientation shown by the third darkest squares is better with cup inclinations around ˜40°-45°, and the orientation shown by the second darkest squares is better with higher cup inclinations (˜50°).

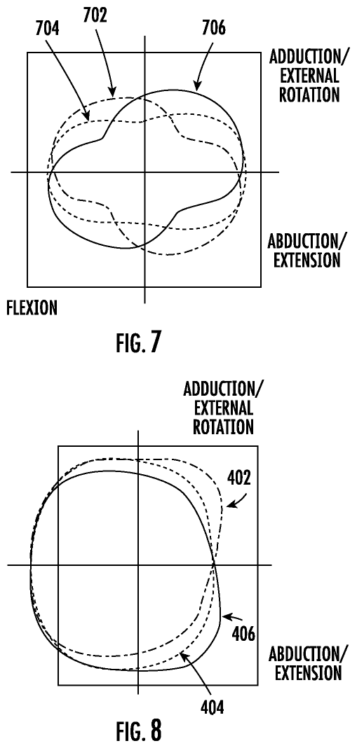

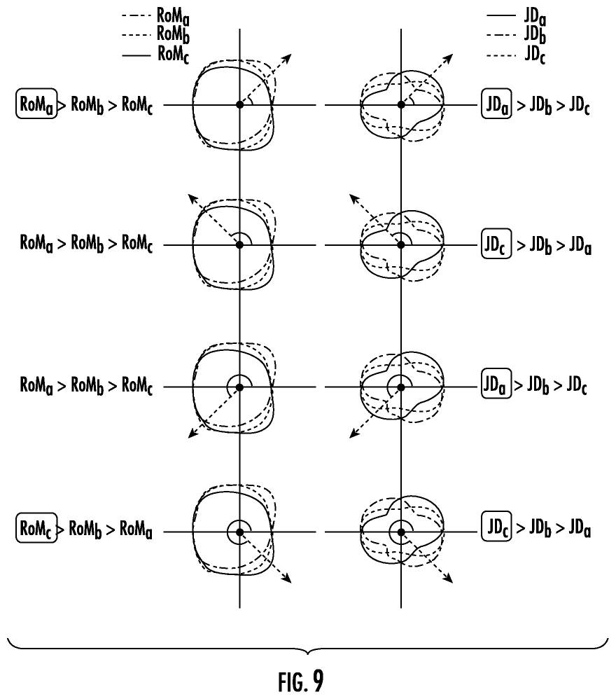

FIG. 7 is a diagram showing the jump distance for various orientations plotted on a coordinate system similar to the one used for showing the range-of-motion, wherein the vertical axis shows rotation of the patient's leg about the X-Axis shown in FIG. 3 and the horizontal axis shows rotation of the patient's leg about the Y-Axis shown in FIG. 3. The profiles for the various orientations of the lip portion of the cup liner 702, 704, 706 show the jump distance profiles for the posterior-inferior (8 o'clock), posterior (9 o'clock) and posterior-superior (10 o'clock) positions respectively. Likewise, FIG. 8 is a diagram showing the range-of-motion for various orientations plotted on the same coordinate system.

The quadrants may be colored to indicate what lip orientation is better (for either range-of-motion or jump distance) for the impingement directions covered by that area. Other indicia (e.g., hatching) may be used to indicate that two orientations are equivalent in that region. So, for example, in the top right quadrant of the range-of-motion plot in FIG. 8, the posterior-superior lip orientation has larger range-of-motion, because the posterior-superior profile 402 is larger than the other two, therefore the background area for this set of impingement directions may be colored a certain color. On the contrary, as shown in FIG. 7, the posterior-inferior lip orientation 706 has larger jump distance profile in that same quadrant because the posterior-inferior jump distance profile 706 is larger than the other two, and therefore the background is colored differently in the jump distance diagram.

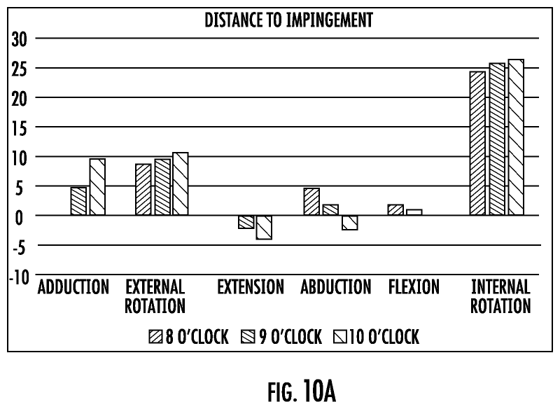

FIG. 9 shows range-of-motion plots on the left and jump distance plots on the right (same two diagrams on each row). The dotted arrow indicates a hypothetical direction of impingement. With reference to the first row, if impingement occurs in the direction of the dotted arrow (first quadrant of the plot), the liner orientation corresponding to the ROMA profiles (posterior-superior) has larger range-of-motion (as indicated in the text to the left of the diagrams), and the liner orientation corresponding to the JD_{A}profiles (posterior-inferior) had larger jump distance (as indicated in the text to the right of the diagrams).

Jump distance is a geometrical property, so it does not change with cup inclination and cup anteversion. However, its relationship with hip physiological ranges of motion will change when changing cup inclination and anteversion. Therefore, the range-of-motion profiles (FIG. 9, left column) and the jump distance profiles (FIG. 9, right column) do not change when changing cup position, but the direction of impingement indicated by the dotted arrow will change. For example, the physiological meaning of the dotted arrow in the third row (third quadrant) might change from flexion with some adduction to flexion with some abduction, depending on cup angles. Changing cup inclination (CI) and cup anteversion (CA) does not modify the ROM_{A}, ROM_{B}, ROM_{C} range-of-motion profiles, but it changes the position and shape of the polygon representing hip kinematics.

In a first example, the plots for range-of-motion and jump distance may be presented to the surgeon, in a format, such as shown in FIG. 5, to assist the surgeon in choosing an optimal placement of the cup and stem portions of the implant. These could be presented pre-operatively. Alternatively, the surgeon may intraoperatively choose the placement of the cup and stem portions of the implant, based on the patient's physiology and anatomy, and may inform the system as to the placement. In response, the system may generate specific plots geared toward the indicated cup inclination, cup anteversion and stem anteversion. The purpose of the diagrams presented to the surgeon are to assist the surgeon in determining the optimal placement of the implant and the lipped portion of the cup liner to maximize the trade-off between range-of-motion and jump distance (i.e., stability). In this first example, the surgeon is presented with the information and makes an informed decision as to the optimal orientation of the implant and the lipped portion of the cup liner based on the information provided.

In the second example, the system may analyze all available data and may recommend an orientation of the implant and the lipped portion of the cup liner. In some features of this example, the recommendation can be provided by a mathematical algorithm, such as a cost optimization algorithm, or by a machine learning model which has been trained on a data set of previous surgical procedures containing data points indicating the placement of the cup and stem portions of the implant as well as the orientation of the lip portion of the cup liner. Alternatively, in other features of the disclosure, the recommendation of one or more possible orientations can be made based on an analysis of the diagrams provided for range-of-motion and jump distance.

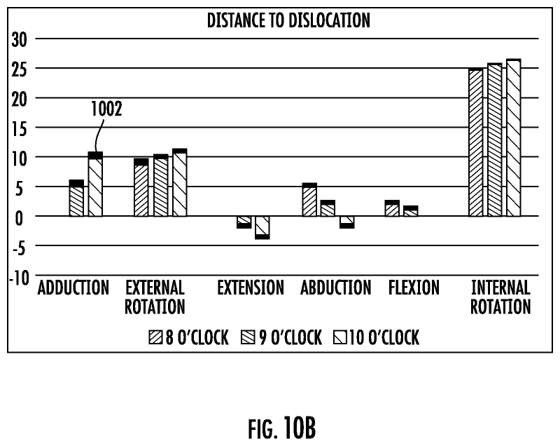

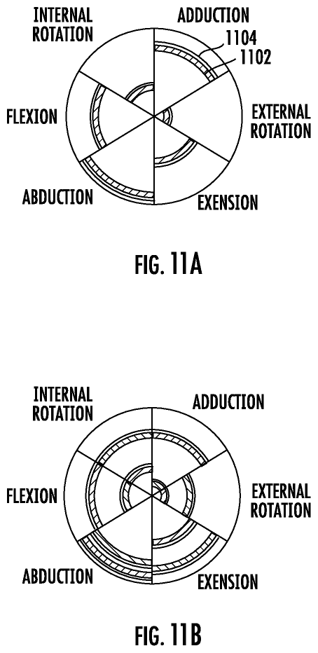

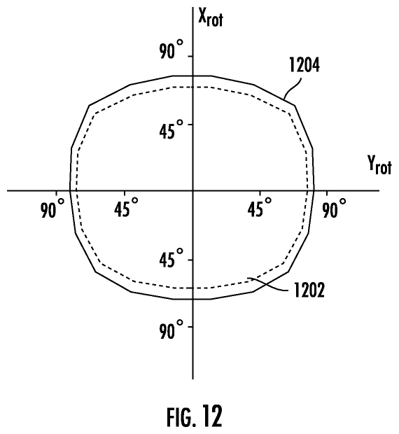

In various alternative examples, the range-of-motion and jump distance may be visualized in any convenient way. The visualizations described above and shown in FIGS. 5-9 should be considered exemplary in nature and the disclosure is not meant to be limited to those visualizations. FIGS. 10A-12 show several alternate visualizations. FIG. 10A is a bar graph showing the distance to impingement as a function of the direction of possible impediments for 3 potential orientations of the lipped portion of the cup liner of the implant (i.e., the 8 o'clock, 9 o'clock and 10 o'clock positions). FIG. 10B shows the distance to dislocation which is, in essence, the distance to impingement plus the jump distance. The jump distance is represented as the solid black areas on the bars in the graph. For example, reference number 1002 in FIG. 10B illustrates the jump distance portion of the bar for the 10 o'clock position of the lipped portion of the cup liner in the abduction direction. FIG. 11A shows the range-of-motion as area 1102 and the jump distance as area 1104 for various directions of possible impingement. For the visualization shown in FIG. 11A, separate visualizations may be provided for different orientations of the lipped portion of the cup liner. FIG. 11B shows a visualization of the range-of-motion and jump distance for two different models of the implant showing a comparison between the range-of-motion and jump distance for each model. FIG. 12 shows yet another visualization of both the range-of-motion and jump distance. Range-of-motion is shown as the inner shape 1202, while the jump distance is shown as shape 1204.

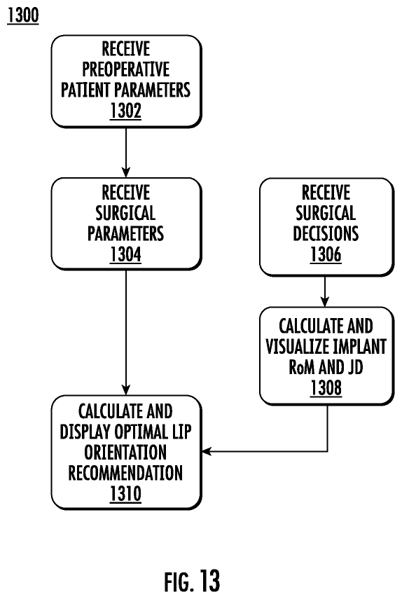

FIG. 13 is a flow diagram of workflow 1300 showing use of the described examples a pre-operative scenario. In the preoperative scenario, the described examples can be used either with a pre-selected cup and step orientation or with the modifiable cup and stem orientation. At step 1302, pre-operative patient parameters are received. These may include, for example, the spinopelvic mobility of the patient. At step 1304, the surgical parameters are received. Here, the surgeon would specify, for example, the surgical approach (e.g., posterior approach, anterior approach). At step 1306, the surgical decisions are received. These may include, for example, implant design. Additionally, in the scenario wherein the cup and stem orientation are pre-selected, the cup orientation and stem orientation would also be received at this step. Based on the received surgical decisions, at step 1308, cup, stem and lip orientations are calculated and visualized. Based on the calculation visualization of the orientations, as well as the received pre-operative patient parameters and surgical parameters, at step 1310, a recommendation is calculated and provided for the optimal orientation of the cup, stem and lip.

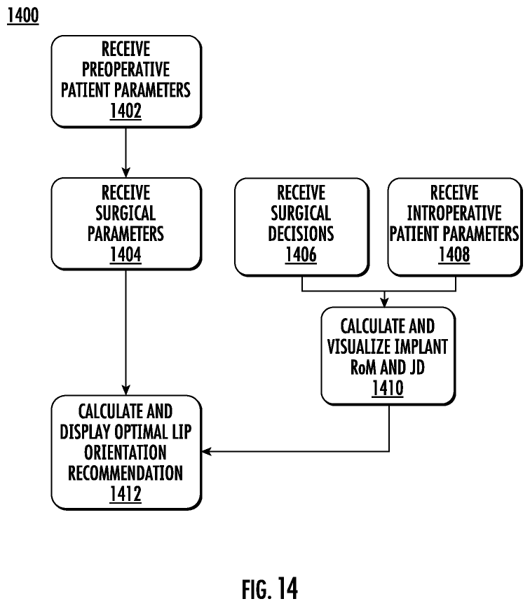

FIG. 14 is a flow diagram of workflow 1400 showing use of the described examples in an intra-operative scenario. As with the pre-operative approach, at step 1402, pre-operative patient parameters are received and at step 1404, the surgical parameters are received. At step 1406, the surgical decisions are received. These may include, for example, implant design, cup orientation and stem orientation. At step 1408, the intra-operative patient parameters are received. These may include, for example, femoral anteversion. Based on the received intra-operative patient parameters and surgical decisions, at step 1410, the implant range of motion and jump distance is calculated and visualized. Based on the calculation visualization of the orientations, as well as the received pre-operative patient parameters and surgical parameters, at step 1410, a recommendation is calculated and provided for the optimal lip orientation.

The systems and methods described herein may be implemented as software application running on a computing system interfaced with a navigated or robotic-assisted surgical platform or, alternatively, as part of the software of the navigated or robotic-assisted surgical platform.

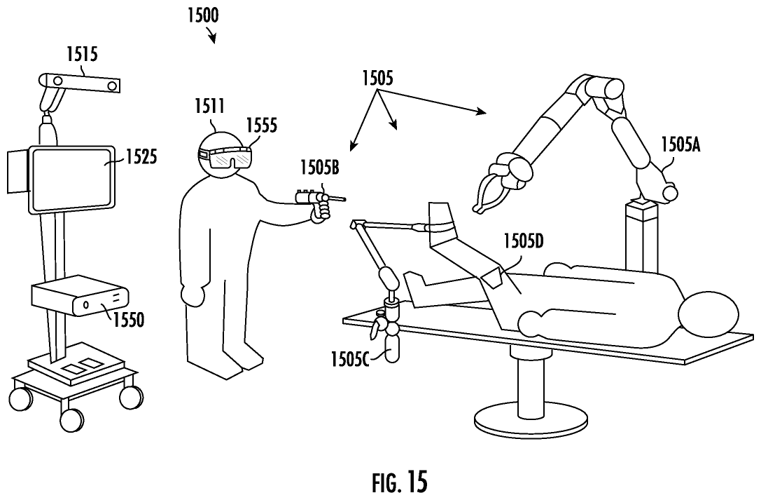

FIG. 15 provides an illustration of an exemplary computer-assisted surgical system 1500, with which the system and method of the present disclosure may be implemented. System 1500 uses computers, robotics, and imaging technology to aid surgeons in performing orthopedic surgery procedures such as total knee arthroplasty (TKA) or total hip arthroplasty (THA). For example, surgical navigation systems can aid surgeons in locating patient anatomical structures, guiding surgical instruments, and implanting medical devices with a high degree of accuracy. Surgical navigation systems 1500 often employ various forms of computing technology to perform a wide variety of standard and minimally invasive surgical procedures and techniques. Moreover, these systems allow surgeons to more accurately plan, track and navigate the placement of instruments and implants relative to the body of a patient, as well as to conduct pre-operative and intra-operative body imaging.

An effector platform 1505 positions surgical tools relative to a patient during surgery. The exact components of the effector platform 1505 will vary, depending on the example employed. For example, for a knee surgery, the effector platform 1505 may include an end effector 1505B that holds surgical tools or instruments during their use. The end effector 1505B may be a handheld device or instrument used by the surgeon (e.g., a NAVIO® hand piece or a cutting guide or jig) or, alternatively, the end effector 1505B can include a device or instrument held or positioned by a robotic arm 1505A.

The effector platform 1505 can include a limb positioner 1505C for positioning the patient's limbs during surgery. One example of a limb positioner 1505C is the SMITH & NEPHEW SPIDER2 system. The limb positioner 1505C may be operated manually by the surgeon or alternatively change limb positions based on instructions received from the surgical computer 1550 (described below).

Resection equipment (not shown in FIG. 15) performs bone or tissue resection using, for example, mechanical, ultrasonic, or laser techniques. Examples of resection equipment include drilling devices, burring devices, oscillatory sawing devices, vibratory impaction devices, reamers, ultrasonic bone cutting devices, radio frequency ablation devices, and laser ablation systems. In some examples, the resection equipment is held and operated by the surgeon during surgery. In other examples, the effector platform 1505 may be used to hold the resection equipment during use.

The effector platform 1505 can also include a cutting guide or jig 1505D that is used to guide saws or drills used to resect tissue during surgery. Such cutting guides 1505D can be formed integrally as part of the effector platform 1505 or robotic arm 1505A or cutting guides can be separate structures that can be removably attached to the effector platform 1505 or robotic arm 1505A. The effector platform 1505 or robotic arm 1505A can be controlled by the system 1500 to position a cutting guide or jig 1505D adjacent to the patient's anatomy in accordance with a pre-operatively or intra-operatively developed surgical plan such that the cutting guide or jig will produce a precise bone cut in accordance with the surgical plan.

The tracking system 1515 uses one or more sensors to collect real-time position data that locates the patient's anatomy and surgical instruments. For example, for TKA procedures, the tracking system 1551 may provide a location and orientation of the end effector 1505B during the procedure. In addition to positional data, data from the tracking system 1515 can also be used to infer velocity/acceleration of anatomy/instrumentation, which can be used for tool control. In some examples, the tracking system 1515 may use a tracker array attached to the end effector 1505B to determine the location and orientation of the end effector 1505B. The position of the end effector 1505B may be inferred based on the position and orientation of the tracking system 1515 and a known relationship in three-dimensional space between the tracking system 1515 and the end effector 1505B. Various types of tracking systems may be used in various examples of the present disclosure including, without limitation, Infrared (IR) tracking systems, electromagnetic (EM) tracking systems, video or image based tracking systems, and ultrasound registration and tracking systems.

The display 1525 provides graphical user interfaces (GUIs) that display images the user interface of the navigated surgical platform, as well as a user interface used to display the plots shown in FIGS. 5-12, as well other information relevant to the surgery. For example, in some examples, the display 1525 overlays image information collected from various modalities (e.g., CT, MRI, X-ray, fluorescent, ultrasound, etc.) collected pre-operatively or intra-operatively to give the surgeon various views of the patient's anatomy as well as real-time conditions. The display 1525 may include, for example, one or more computer monitors. As an alternative or supplement to the display 1525, one or more members of the surgical staff may wear an augmented reality (AR) Head Mounted Device (HMD). For example, in FIG. 15 the surgeon 1511 is wearing an AR HMD 1555 that may, for example, overlay pre-operative image data on the patient or provide surgical planning suggestions. Various example uses of the AR HMD 1555 in surgical procedures are detailed in the sections that follow.

Surgical computer 1550 provides control instructions to various components of system 1500, collects data from those components, and provides general processing for various data needed during surgery. In some examples, the surgical computer 1550 is a general purpose computer. In other examples, the surgical computer 1550 may be a parallel computing platform that uses multiple central processing units (CPUs) or graphics processing units (GPU) to perform processing. In some examples, the surgical computer 1550 is connected to a remote server over one or more computer networks (e.g., the Internet). The remote server can be used, for example, for storage of data or execution of computationally intensive processing tasks.

In various examples, the systems and methods of the present disclosure may be implemented as a software application executing on surgical computer 1550 integrated with or separate from the software controlling the navigated surgical platform. Also, in various examples, systems and methods of the present disclosure may be implemented as a software application executing on a computing platform other than surgical computer 1550, such as a laptop or tablet computing device. The computing device executing systems and methods of the present disclosure may be in communication with surgical computer 1550.

Various examples of a system and method have been described herein to provide a surgeon performing total hip replacement procedures with a tool for visualizing the orientation of the lipped portion of the cup liner based on the positioning of the cup and stem portions of the implant. In some examples a pre-operative portion may be shown to provide the surgeon with different options as far as cup and stem placement and their impact on various orientations of the lipped portion of the cup liner. In a second example, the system provides an optimal recommendation as to the orientation of the cup liner based on indicated positions of the cup and stem portions of the implant.

As would be realized by one of skill in the art, the present disclosure has been explained in terms of a posterior surgical approach. However, the system is equally applicable to surgeries utilizing anterior or lateral surgical approaches, which would result in different stability provided by the soft tissues surrounding the hip.

Previous methods for selecting the orientation of the cup range from educated guessing to intuitive conclusions based on previous experience of the surgeon and the surgical approach. The system and method disclosed herein provides a heretofore unavailable tool for the surgeon to improve and inform the selection of the orientation of the lipped portion of the cup liner.

We claim:

1. A method comprising:
visualizing a range-of-motion for a plurality of orientations of a hip implant based on various combinations of placement options for a cup portion and a stem portion of the implant; and
visualizing a jump distance for a plurality of orientations of the implant.

2. The method of claim 1, further comprising:
providing 2D or 3D visualizations of the implant's range-of-motion and jump distance profiles for various possible directions of impingement.

3. The method of claim 2, further comprising:
providing a visualization of a desired range-of motion of the patient's leg as an overlay on the range-of-motion.

4. The method of claim 3, further comprising:
visualizing the range-of-motion by plotting one or more profiles representing one or more ranges-of-motion on a coordinate system having a vertical axis defining rotation of the patient's leg around an X-axis and a horizontal axis defining rotation of the patient's leg around a Y-axis.

5. The method of claim 4, wherein the ranges-of-motion of a patient's leg are visualized as a polygon overlaid on the coordinate system showing the kinematics of a hip based on a positioning of the implant, wherein the plurality of profiles represent different orientations of the implant.

6. The method of claim 5, further comprising:
determining acceptable orientations of the implant by determining whether the visualization of the ranges-of-motion of the patient's leg representing the kinematics of the hip extends outside of one or more of the profiles representing the range-of-motion.

7. The method of claim 6, further comprising:
determining the positioning of the cup portion and stem portion of the implant for various cup inclinations, cup anteversions and stem anteversions.

8. The method of claim 7, wherein visualizing the jump distance comprises:
plotting a plurality of profiles representing the jump distance on a coordinate system having a vertical axis corresponding to rotations of the patient's leg about an anterior-posterior axis of a cup portion of the implant and a horizontal axis corresponding to rotations of the patient's leg about a medial-lateral axis of the cup axis of the cup portion of the implant.

9. The method of claim 8, further comprising:
selecting, for each quadrant of the coordinate system, a preferred range-of-motion and jump distance profile such that the range-of-motion and jump distance are maximized for possible directions of impingement.

10. The method of claim 9, comprising:
receiving an input of a positioning of the implant.

11. The method of claim 10, further comprising:
receiving an indication of a cup anteversion, a stem anteversion and a cup inclination; and
providing a visualization of effect of the placement of a lipped portion of the cup liner on the range-of-motion and jump distance based on the received indications.

12. A system comprising:
a processor; and
software that, when execute by the processor, causes the system to implement the method of claim 11.

13. The method of claim 11, further comprising:
providing a plurality of surgical recommendations based on various possible positionings of the implant and other pre-operative or intra-operative patient or surgical parameters.

14. The method of claim 13, further comprising:
providing a recommendation, based on the received input, for orientation of the lipped portion of the cup liner.

15. The method of claim 14, further comprising:
analyzing the indicated implant positioning; and
providing an optimal recommendation of the orientation of the lipped portion of the cup liner via a mathematical algorithm or a machine learning model.

16. A system comprising:
a processor; and
software that, when execute by the processor, causes the system to implement the method of claim 15.
